(19) United States
(12) Patent Application Publication
(10) Pub. No.: US 2024/0277258 A1
(43) Pub. Date: Aug. 22, 2024

(54) SYSTEM FOR GENERATING DRUG PRESCRIPTION RECOMMENDATIONS FOR IMPROVING ATTENTION, AND METHOD OF USE THEREOF

(71) Applicant: REVIBE TECHNOLOGIES, INC., Wake Forest, NC (US)

(72) Inventors: Richard BRANCACCIO, Wake Forest, NC (US); Joseph KOZIAK, Phoenix, AZ (US); Christopher GUIDRY, Chapel Hill, NC (US); Lindsay AYEARST, Markham (CA); David VAUGHN, Decatur, GA (US); Zhenzhen ZHANG, Raleigh, NC (US)

(21) Appl. No.: 18/571,001

(22) PCT Filed: Jun. 17, 2022
(86) PCT No.: PCT/US2022/034001

§ 371 Date: Dec. 15, 2023

Related U.S. Application Data
(60) Provisional application No. 63/279,038, filed on Nov. 12, 2021.
(60) Provisional application No. 63/212,564, filed on Jun. 18, 2021.

(51) Int. Cl. A61B 5/11; A61B 5/00; G16H 15/00; G16H 20/10

(52) U.S. Cl. CPC A61B 5/11; A61B 5/4088; A61B 5/6802; A61B 5/725; A61B 5/7267; G16H 15/00; G16H 20/10; A61B 2562/0219

(57) ABSTRACT

A method for generating a report on medication prescription for treating a condition of a subject that affects attention: it includes receiving movement data of said patient: categorizing the movement data: receiving profile information of said subject: transmitting a query to a database of profiles of subjects having received a prescription for improving attention, wherein the query includes a plurality of criteria based on the profile information of the subject and the fidgeting data: receiving the profiles of subjects having received a prescription for improving attention corresponding to the query: and generating a prescription recommendation in accordance with one or more prescriptions indicated for the received profiles of subjects.

The present application claims priority from U.S. provisional patent application No. 63/212,564 filed on Jun. 18, 2021, incorporated herein by reference, and U.S. provisional patent application No. 63/279,038 filed on Nov. 12, 2021, incorporated herein by reference.

### TECHNICAL FIELD

The present disclosure relates to improving attention and focus of a subject, and more particularly to the treatment of disorders that impede attention and focus of the subject.

### BACKGROUND

Conditions such as Attention Deficit Hyperactivity Disorder (ADHD) impede a subject's ability to concentrate for prolonged periods when performing cognitive tasks.

When physicians prescribe medication to reduce the symptoms of ADHD, the physician prescribes one or more of a selection of drugs based on limited data on the subject, such as age, gender, the subject's recollections as well as that of close ones (e.g. parents, significant other, teacher, etc.) and general condition when seen by the physician. However, the physician does not have the capacity to monitor the patient for a prolonged period nor in a setting other that in the physician's office. As such, the physician's assessment of the patient is limited to what is observed in a non-ordinary setting, namely at the physician's office. The physician cannot monitor the patient at home, in class, etc. This limited information on the patient's behavior impedes the physician in accurately prescribing an appropriate drug, not to mention the optimal dosage.

Moreover, when the physician selects a drug or dosage, they base themselves on their own medical experience and current medical guidelines. As such, the physician may be limited to their own history with their own finite number of patients, influencing their decision. This pool of patients that serves the physician as a benchmark for prescribing a drug to a current patient is limited.

The limited information available to the physician to make a decision as to which drug to prescribe often results in a prescription with mixed results, where the patient may suffer from undesirable side effects from the drug while only seeing a limited, if at that, improvement in focus and attention when performing cognitive tasks. Therefore, it would be advantageous to avail the physician with more information on the patient's behavior, or factor in this additional information when prescribing a drug, as well as guide the drug prescription by the physician based on experience gathered from a larger basing of patients, that can assist the physician in prescribing a drug to a patient for improving focus and attention (e.g. treating ADHD).

### SUMMARY

U.S. Pat. No. 10,624,590 describes a wristband for generating attention prompting signals (e.g. vibrations) for improving the focus of a wearer, where the signals remind the wearer to focus on a task. A similar device can also be used to monitor the wearer during the day (as the wearer performs day-to-day activities such as going to class, doing sports, etc.) by collecting movement data on the subject, where the movement data can be analyzed to determine an amount of fidgeting of the patient. The fidgeting can provide information on a level of attention and focus of the patient, where a higher amount of undesirable fidgeting can be an indicator that the subject is having difficulty focusing on a cognitive task.

This movement data and/or feedback data (self-reports from a user regarding a legal of attention of the user) generated throughout the day by the device provides a more encompassing portrait of the behavior of the patient, as well as an indicator as to the level of focus and attention of the patient as the patient performs cognitive tasks. When this collected movement data is compared with similar data of other subjects wearing a device or subject with a condition hampering their focus and attention (where these subjects have been administered a drug), the combination of this more thorough data on the subject of interest and a greater pool of subjects to compare the subject of interest to for purposes of prescribing a drug, result in generating of recommendations for a physician that can assist the physician in more accurately prescribing a drug that will be effective for the subject of interest.

A broad aspect is a method for generating a report on medication prescription for treating a condition of a subject that affects attention. It includes receiving movement data of said patient, whereby the movement data was collected over a given time period by one or more motion sensors (that can detect vibrations) worn by the patient; categorizing the movement data as associated with a standing position or a sitting position of said subject through a previously train artificial intelligence model, and wherein movement data associated with the seated position is further categorized to determine fidgeting through the previously trained artificial intelligence model to generate fidgeting data associated with a lack of attention, wherein the previously trained artificial intelligence model is a movement classifying engine that is trained with learning data including previous movement data that is matched with one or more labels associated with a standing position or a sitting position, and further matched, when the movement data is associated with a sitting position, with a label for a presence or absence of fidgeting; receiving profile information of said subject, including an age value and a gender value; transmitting a query to a database of profiles of subjects having received a prescription for improving attention, wherein the query includes a plurality of criteria based on the profile information of the subject and the fidgeting data; receiving the profiles of subjects having received a prescription for improving attention corresponding to the query; and generating a prescription recommendation in accordance with one or more prescriptions indicated for the received profiles of subjects.

In some embodiments, the received movement data may be filtered using a noise filter prior to receipt.

In some embodiments, the condition of the subject may be attention deficit hyperactivity disorder.

In some embodiments, movement data associated with the standing position may be further analyzed to determine a number of steps and to categorize the standing position movement data as being attributable to a “running state” or a “walking state”, and further calculating a value for calorie consumption of the subject from the number of steps and a calorie consumption rate value associated with the running state or the walking state, and wherein the plurality of criteria for the query may be based on the value for calorie consumption.

In some embodiments, after the receiving the profiles, the method may include determining a number of profiles received from the query; and comparing the number of profiles to a profile threshold value; if the number of profiles is less than the profile threshold value, generating a revised query removing one or more criteria of the plurality of conditions, and transmitting the revised query to the database of profiles.

In some embodiments, the fidgeting data may include one or more labels for categories of fidgeting including types of fidgeting.

In some embodiments, the method may include receiving at least one of parent reports of the subject, teacher reports of the subject and personal reports of the subject, for providing reported information on the subject during a time period, and wherein the plurality of criteria for the query is based on the reported information to further analyze the attention of the subject.

In some embodiments, the movement data may be collected using a wristband worn by the subject, the wristband including one or more accelerometers as the motion sensors.

In some embodiments, the prescription recommendation may include a dosage.

Another broad aspect is a system for generating a report on medication prescription for treating a condition of a subject that affects attention. The system includes a processor; memory comprising program code that, when executed by the processor, cause the processor to receive movement data of said subject, whereby the movement data was generated over a given time period by one or more motion sensors worn by the subject; categorize the movement data as associated with a standing position or a sitting position of said subject through a previously train artificial intelligence model, and wherein movement data associated with the seated position is further categorized to determine fidgeting through the previously trained artificial intelligence model to generate fidgeting data associated with a lack of attention, wherein the previously trained artificial intelligence model is a movement classifying engine that is trained with learning data including previous movement data that is matched with one or more labels associated with a standing position or a sitting position, and further matched, when the movement data is associated with a sitting position, with a label for a presence or absence of fidgeting; receive profile information of said subject, including an age value and a gender value; transmit a query to a database of profiles of subjects having received a prescription for improving attention, wherein the query includes a plurality of criteria based on the profile information of the subject and the fidgeting data; receive the profiles of subjects having received a prescription for improving attention corresponding to the query; and generate a prescription recommendation in accordance with one or more prescriptions indicated in the received profiles of subjects.

Another broad aspect is a method for generating a report on medication prescription for treating a condition of a subject that affects attention of the subject. The method includes receiving movement data of the subject, whereby the movement data was collected over a given time period by one or more motion sensors worn by the subject; categorizing the movement data as associated with fidgeting movement or non-fidgeting movement using a previously trained artificial intelligence model to generating fidgeting data that is indicative as to a lack of focus of the subject, wherein the previously trained artificial intelligence model is a movement classifying engine that is trained with learning data including samples of movement data that is matched with labels for fidgeting movement or non-fidgeting movement; receiving characteristic data of said subject, including an age value and a gender value; transmitting a query to a database of profile data structures of subjects having received a prescription for improving attention, wherein the query includes a plurality of criteria based on the characteristic data of the subject and the fidgeting data; receiving the profile data structure information of subjects having received a prescription for improving attention corresponding to the query; and generating a prescription recommendation in accordance with one or more prescriptions indicated in the received profile data structures of subjects.

In some embodiments, the method may include, prior to the categorizing the movement data as associated with fidgeting movement or non-fidgeting movement, categorizing the movement data as associated with a standing position or a sitting position of the subject using the previously trained artificial intelligence model, and wherein the categorizing the movement data as associated with fidgeting movement or non-fidgeting movement is performed on the movement data associated with the seated position, and wherein the previously trained artificial intelligence model is a movement classifying engine that is further trained with learning data including samples of movement data that is matched with labels for sitting position or a standing position.

In some embodiments, the movement data associated with the standing position may be further analyzed to determine a number of steps and to categorize the standing position movement data as being attributable to a “running state” or a “walking state”, and further calculating a value for calorie consumption of the subject from the number of steps and a calorie consumption rate value associated with the running state or the walking state, and wherein the plurality of criteria for the query is based on the value for calorie consumption.

In some embodiments, the received movement data may have been filtered using a noise filter prior to receipt.

In some embodiments, the condition of the subject may be attention deficit hyperactivity disorder.

In some embodiments, after the receiving of the profile data structure information, the method may include: determining a number of profiles received from the query; comparing the number of profiles to a profile threshold value; and if the number of profiles is less than the profile threshold value, generating a revised query removing one or more criteria of the plurality of conditions, and transmitting the revised query to the database of profiles.

In some embodiments, the movement data with a label for fidgeting movement may be further categorized by associating the movement data with the label for fidgeting movement with a label for a category of fidgeting movement selected from one or more types of fidgeting using the previously trained artificial intelligence model, wherein the previously trained artificial intelligence model is a movement classifying engine that is further trained with learning data including samples of fidgeting movement data that is matched with labels for one or more of types of fidgeting.

In some embodiments, the method may include receiving at least one of parent reports of the subject, teacher reports of the subject and personal reports of the subject, for providing reported information on the subject during a time period, and wherein the plurality of criteria for the query is based on the reported information to further analyze the attention of the subject.

In some embodiments, the collected movement data may have been generated using a wristband worn by the subject, the wristband including one or more accelerometers as the movement sensors.

In some embodiments, the prescription recommendation may include a dosage.

Another broad aspect is a system for generating a report on medication prescription for treating a condition of a subject that affects attention. The system includes a processor; memory comprising program code that, when executed by the processor, cause the processor to receive movement data of the subject, whereby the movement data was collected over a given time period by one or more movement sensors worn by the subject; categorize the movement data as associated with fidgeting movement or non-fidgeting movement using a previously trained artificial intelligence model to generating fidgeting data (or unhelpful fidgeting data as described herein) that may be indicative as to a lack of attention of the subject, wherein the previously trained artificial intelligence model is a movement classifying engine that is trained with learning data including samples of movement data that is matched with labels for fidgeting movement or non-fidgeting movement; receive characteristic data of said subject, including an age value and a gender value; transmit a query to a database of profile data structures of subjects having received a prescription for improving attention, wherein the query includes a plurality of criteria based on the characteristic data of the subject and the fidgeting data; receive the profile data structure information of subjects having received a prescription for improving attention corresponding to the query; and generate a prescription recommendation in accordance with one or more prescriptions indicated in the received profile data structures of subjects.

In some embodiments, the program code may include program code that, when executed by the processor, causes the processor to, prior to the categorizing the movement data as associated with fidgeting movement or non-fidgeting movement, categorize the movement data as associated with a standing position or a sitting position of the subject using the previously trained artificial intelligence model, and wherein the categorizing the movement data as associated with fidgeting movement or non-fidgeting movement is performed on the movement data associated with the seated position, and wherein the previously trained artificial intelligence model is a movement classifying engine that is further trained with learning data including samples of movement data that is matched with labels for sitting position or a standing position.

In some embodiments, the program code may include program code that, when executed by the processor, causes the processor to further analyze the movement data associated with the standing position to determine a number of steps and to categorize the standing position movement data as being attributable to a “running state” or a “walking state”, and further calculating a value for calorie consumption of the subject from the number of steps and a calorie consumption rate value associated with the running state or the walking state, and wherein the plurality of criteria for the query is based on the value for calorie consumption.

In some embodiments, the received movement data may have been filtered using a noise filter prior to receipt.

In some embodiments, the condition of the subject may be attention deficit hyperactivity disorder.

In some embodiments, the program code may include program code that, when executed by the processor, causes the processor to, after the receiving of the profile data structure information, determine a number of profiles received from the query; compare the number of profiles to a profile threshold value; and if the number of profiles is less than the profile threshold value, generate a revised query removing one or more criteria of the plurality of conditions, and transmitting the revised query to the database of profiles.

In some embodiments, the program code may include program code that, when executed by the processor, causes the processor to further categorize the movement data with a label for fidgeting movement by associating the movement data with the label for fidgeting movement with a label for a category of fidgeting movement selected from one or more types of fidgeting using the previously trained artificial intelligence model, wherein the previously trained artificial intelligence model is a movement classifying engine that is further trained with learning data including samples of fidgeting movement data that is matched with labels for one or more of types of fidgeting.

In some embodiments, the program code may include program code that, when executed by the processor, causes the processor to receive at least one of parent reports of the subject, teacher reports of the subject and personal reports of the subject, for providing reported information on the subject during a time period, and wherein the plurality of criteria for the query is based on the reported information to further analyze the attention of the subject.

In some embodiments, the collected movement data may have been generated using a wristband worn by the subject, the wristband including one or more accelerometers as the motion sensors.

In some embodiments, the system may include the wristband.

In some embodiments, the prescription recommendation may include a dosage.

Another broad aspect is a non-transitory storage medium that, when executed by a processor, causes the processor to receive movement data of the subject, whereby the movement data was collected over a given time period by one or more movement sensors worn by the subject; categorize the movement data as associated with fidgeting movement or non-fidgeting movement using a previously trained artificial intelligence model to generating fidgeting data that is indicative as to a lack of attention of the subject, wherein the previously trained artificial intelligence model is a movement classifying engine that is trained with learning data including samples of movement data that is matched with labels for fidgeting movement or non-fidgeting movement; receive characteristic data of said subject, including an age value and a gender value; transmit a query to a database of profile data structures of subjects having received a prescription for improving attention, wherein the query includes a plurality of criteria based on the characteristic data of the subject and the fidgeting data; receive the profile data structure information of subjects having received a prescription for improving attention corresponding to the query; and generate a prescription recommendation in accordance with one or more prescriptions indicated in the received profile data structures of subjects.

Another broad aspect is a method for generating a report on medication prescription for treating a condition of a subject that affects attention of the subject, comprising receiving movement data of the subject, whereby the movement data was collected over a given time period by one or more motion sensors worn by the subject; categorizing the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement; receiving characteristic data of said subject, including an age value and a gender value; transmitting a query to a database of profile data structures of subjects having received a prescription for improving at least one attention, wherein the query includes a plurality of criteria based on the characteristic data of the subject and the fidgeting data; receiving the profile data structure information of subjects having received a prescription for improving attention corresponding to the query; and generating a prescription recommendation in accordance with one or more prescriptions indicated in the received profile data structures of subjects.

In some embodiments, profile data structures of the database may be of subjects that have received a prescription for treating attention of the subject, and where the prescription has improved the attention of the subject.

In some embodiments, the method may include, prior to the categorizing the movement data as associated with fidgeting movement or non-fidgeting movement, categorizing the movement data as associated with a standing position or a sitting position of the subject using the previously trained artificial intelligence model, and wherein the categorizing the movement data as associated with fidgeting movement or non-fidgeting movement is performed on the movement data associated with the seated position, and wherein the previously trained artificial intelligence model is a movement classifying engine that is further trained with learning data including samples of movement data that is matched with labels for sitting position or a standing position.

In some embodiments, movement data associated with the standing position may be further analyzed to determine a number of steps and to categorize the standing position movement data as being attributable to a “running state” or a “walking state”, and further calculating a value for calorie consumption of the subject from the number of steps and a calorie consumption rate value associated with the running state or the walking state, and wherein the plurality of criteria for the query is based on the value for calorie consumption.

In some embodiments, the method may include receiving feedback from the subject on a level of attention of a subject regarding a task; correlating the received feedback from the subject with the movement data that is labelled as fidgeting movement; and categorizing movement data that is labelled as fidgeting movement, following the correlation, as unhelpful or helpful fidgeting movement based on an indication that the subject is on-task or off-task from the received feedback.

In some embodiments, the received movement data may be filtered using a noise filter prior to receipt.

In some embodiments, the condition of the subject may be attention deficit hyperactivity disorder.

In some embodiments, after the receiving of the profile data structure information, the method may include determining a number of profiles received from the query; comparing the number of profiles to a profile threshold value; and if the number of profiles is less than the profile threshold value, generating a revised query removing one or more criteria of the plurality of conditions, and transmitting the revised query to the database of profiles.

In some embodiments, movement data with a label for fidgeting movement may be further categorized by associating the movement data with the label for fidgeting movement with a label for a category of fidgeting movement selected from one or more types of fidgeting types of fidgeting using the previously trained artificial intelligence model, wherein the previously trained artificial intelligence model is a movement classifying engine that is further trained with learning data including samples of fidgeting movement data that is matched with labels for one or more of types of fidgeting types of fidgeting.

In some embodiments, the method may include receiving at least one of parent reports of the subject, teacher reports of the subject and personal reports of the subject, for providing reported information on the subject during a time period, and wherein the plurality of criteria for the query is based on the reported information to further analyze the attention of the subject.

In some embodiments, the collected movement data may have been generated using a wristband worn by the subject, the wristband including one or more accelerometers as the motion sensors.

In some embodiments, the prescription recommendation may include a dosage.

Another broad aspect is a system for generating a report on medication prescription for treating a condition of a subject that affects attention. The system includes a processor; memory comprising program code that, when executed by the processor, cause the processor to receive movement data of the subject, whereby the movement data was collected over a given time period by one or more motion sensors worn by the subject; categorize the movement data as associated with fidgeting movement or non-fidgeting movement using a previously trained artificial intelligence model to generating fidgeting data that is indicative as to a lack of attention of the subject, wherein the previously trained artificial intelligence model is a movement classifying engine that is trained with learning data including samples of movement data that is matched with labels for fidgeting movement or non-fidgeting movement; receive characteristic data of said subject, including an age value and a gender value; transmit a query to a database of profile data structures of subjects having received a prescription for improving attention, wherein the query includes a plurality of criteria based on the characteristic data of the subject and the fidgeting data; receive the profile data structure information of subjects having received a prescription for improving attention corresponding to the query; and generate a prescription recommendation in accordance with one or more prescriptions indicated in the received profile data structures of subjects.

In some embodiments, the program code may include program code that, when executed by the processor, causes the processor to, prior to the categorizing the movement data as associated with fidgeting movement or non-fidgeting movement, categorize the movement data as associated with a standing position or a sitting position of the subject using the previously trained artificial intelligence model, and wherein the categorizing the movement data as associated with fidgeting movement or non-fidgeting movement is performed on the movement data associated with the seated position, and wherein the previously trained artificial intelligence model is a movement classifying engine that is further trained with learning data including samples of movement data that is matched with labels for sitting position or a standing position.

In some embodiments, the program code may include program code that, when executed by the processor, causes the processor to further analyze the movement data associated with the standing position to determine a number of steps and to categorize the standing position movement data as being attributable to a “running state” or a “walking state”, and further calculating a value for calorie consumption of the subject from the number of steps and a calorie consumption rate value associated with the running state or the walking state, and wherein the plurality of criteria for the query is based on the value for calorie consumption.

In some embodiments, the received movement data may be filtered using a noise filter prior to receipt.

In some embodiments, the condition of the subject may be attention deficit hyperactivity disorder.

In some embodiments, the program code may include program code that, when executed by the processor, causes the processor to, after the receiving of the profile data structure information determine a number of profiles received from the query; compare the number of profiles to a profile threshold value; and if the number of profiles is less than the profile threshold value, generate a revised query removing one or more criteria of the plurality of conditions, and transmitting the revised query to the database of profiles.

In some embodiments, the program code may include program code that, when executed by the processor, causes the processor to further categorize the movement data with a label for fidgeting movement by associating the movement data with the label for fidgeting movement with a label for a category of fidgeting movement selected from one or more types of fidgeting using the previously trained artificial intelligence model, wherein the previously trained artificial intelligence model is a movement classifying engine that is further trained with learning data including samples of fidgeting movement data that is matched with labels for one or more of types of fidgeting.

In some embodiments, the program code may include program code that, when executed by the processor, causes the processor to receive at least one of parent reports of the subject, teacher reports of the subject and personal reports of the subject, for providing reported information on the subject during a time period, and wherein the plurality of criteria for the query is based on the reported information to further analyze the attention of the subject.

In some embodiments, the collected movement data may have been generated using a wristband worn by the subject, the wristband including one or more accelerometers as the motion sensors.

In some embodiments, the system may include the wristband.

In some embodiments, the prescription recommendation may include a dosage.

In some embodiments, the memory may include program code that, when executed by the processor, cause the processor to receive feedback from the subject on a level of attention of a subject regarding a task; correlate the received feedback from the subject with the movement data that is labeled as fidgeting movement; and categorize movement data that is labeled as fidgeting movement, following the correlation, as unhelpful or helpful fidgeting movement based on an indication that the subject is on-task or off-task from the received feedback.

Another broad aspect is non-transitory storage medium that, when executed by a processor, causes the processor to receive movement data of the subject, whereby the movement data was collected over a given time period by one or more motion sensors worn by the subject; categorize the movement data as associated with fidgeting movement or non-fidgeting movement using a previously trained artificial intelligence model to generating fidgeting data that is indicative as to a lack of attention of the subject, wherein the previously trained artificial intelligence model is a movement classifying engine that is trained with learning data including samples of movement data that is matched with labels for fidgeting movement or non-fidgeting movement; receive characteristic data of said subject, including an age value and a gender value; transmit a query to a database of profile data structures of subjects having received a prescription for improving attention, wherein the query includes a plurality of criteria based on the characteristic data of the subject and the fidgeting data; receive the profile data structure information of subjects having received a prescription for improving attention corresponding to the query; and generate a prescription recommendation in accordance with one or more prescriptions indicated in the received profile data structures of subjects.

A broad aspect of the present disclosure is a method for identifying fidgeting of a subject from movement data gathered by a wearable device while worn by a subject, the wearable device including at least one of one or more accelerometers and one or more gyroscopes, the fidgeting related to a level of attention of the subject. The method includes receiving movement data of the subject comprising one or more of force magnitude information, force direction information and angular velocity information generated by the at least one of one or more accelerometers and one or more gyroscopes; categorizing the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement, associated with a level of attention of the subject.

In some embodiments, the method may include filtering the movement data as being associated with a sitting state of the subject, wherein the movement data associated that is categorized as associated with fidgeting movement or non-fidgeting movement may be the movement data associated with the sitting state.

In some embodiments, the filtering may be performed using a trained long short-term memory artificial intelligence model.

In some embodiments, the method may include further categorizing the movement data with a label of fidgeting movement as a sub-type of fidgeting using a previously trained artificial intelligence model to generate fidgeting data that is indicative as to a lack of attention of the subject, wherein the previously trained artificial intelligence model is a movement classifying engine that is trained with learning data including samples of fidgeting-labeled movement data that is matched with labels for sub-categories of fidgeting movement.

In some embodiments, the labels for sub-categories of fidgeting movement may include drumming and tapping.

In some embodiments, the method may include: receiving feedback from the subject on a level of attention of a subject regarding a task; correlating the received feedback from the subject with the movement data that is labeled as fidgeting movement; and categorizing movement data that is labelled as fidgeting movement, following the correlation, as unhelpful or helpful fidgeting movement based on an indication that the subject is on-task or off-task from the received feedback.

In some embodiments, the received movement data may be filtered using a noise filter prior to receipt.

Another broad aspect of the present disclosure a method for identifying fidgeting of a subject from movement data gathered by a wearable device while worn by a subject, the wearable device including at least one of one or more accelerometers and one or more gyroscopes, the fidgeting related to a level of attention of the subject. The method may include receiving movement data of the subject comprising one or more of force magnitude information, force direction information and angular velocity information generated by the at least one of one or more accelerometers and one or more gyroscopes; categorizing the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement; receiving user input from the wearable device relating to attention of the subject; correlating time of the user input with time of the movement data that is categorized as fidgeting movement; and further categorizing the fidgeting movement into unhelpful fidgeting or helpful fidgeting as a function of the user input that has the time of the user input correlated with the corresponding time of the movement data of the categorized fidgeting movement.

In some embodiments, the method may include filtering the movement data as being associated with a sitting state of the subject, wherein the movement data associated that is categorized as associated with fidgeting movement or non-fidgeting movement is the movement data associated with the sitting state.

In some embodiments, the user input may be indicative of the subject being on-task or off-task.

In some embodiments, the received movement data may be filtered using a noise filter prior to receipt.

In some embodiments, the method may include filtering the received movement data using a noise filter.

Another broad aspect is a system for identifying fidgeting of a subject from movement data gathered by a wearable device while worn by a subject, the wearable device including at least one of one or more accelerometers and one or more gyroscopes, the fidgeting related to a level of attention of the subject. The system includes a processor; memory comprising program code that, when executed by the processor, causes the processor to receive movement data of the subject comprising one or more of force magnitude information, force direction information and angular velocity information generated by the at least one of one or more accelerometers and one or more gyroscopes; categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement, associated with a level of attention of the subject.

In some embodiments, the memory may include program code that, when executed by the processor, causes the processor to filter the movement data as being associated with a sitting state of the subject, wherein the movement data associated that is categorized as associated with fidgeting movement or non-fidgeting movement is the movement data associated with the sitting state.

In some embodiments, the filtering may include performing using a trained long short-term memory artificial intelligence model.

In some embodiments, the memory may include program code that, when executed by the processor, causes the processor to further categorize the movement data with a label of fidgeting movement as a sub-type of fidgeting using a previously trained artificial intelligence model to generate fidgeting data that is indicative as to a lack of attention of the subject, wherein the previously trained artificial intelligence model is a movement classifying engine that is trained with learning data including samples of fidgeting-labeled movement data that is matched with labels for sub-categories of fidgeting movement.

In some embodiments, the labels for sub-categories of fidgeting movement may include drumming and tapping.

In some embodiments, the memory may include program code that, when executed by the processor, causes the processor to receive feedback from the subject on a level of attention of a subject regarding a task; correlate the received feedback from the subject with the movement data that is labelled as fidgeting movement; and categorize movement data that is labelled as fidgeting movement, following the correlation, as unhelpful or helpful fidgeting movement based on an indication that the subject is on-task or off-task from the received feedback.

In some embodiments, the received movement data may be filtered using a noise filter prior to receipt.

Another broad aspect is a system for identifying fidgeting of a subject from movement data gathered by a wearable device while worn by a subject, the wearable device including at least one of one or more accelerometers and one or more gyroscopes, the fidgeting related to a level of attention of the subject: a processor; memory comprising program code that, when executed by the processor, cause the processor to receive movement data of the subject comprising one or more of force magnitude information, force direction information and angular velocity information generated by the at least one of one or more accelerometers and one or more gyroscopes; categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement; receive user input from the wearable device relating to attention of the subject; correlate time of the user input with time of the movement data that is categorized as fidgeting movement; and further categorize the fidgeting movement into unhelpful fidgeting or helpful fidgeting as a function of the user input that has the time of the user input correlated with the corresponding time of the movement data of the categorized fidgeting movement.

In some embodiments, the memory may include program code that, when executed by the processor, causes the processor to filter the movement data as being associated with a sitting state of the subject, wherein the movement data associated that is categorized as associated with fidgeting movement or non-fidgeting movement is the movement data associated with the sitting state.

In some embodiments, the user input may be indicative of the subject being on-task or off-task.

In some embodiments, the received movement data may be filtered using a noise filter prior to receipt.

In some embodiments, the memory may include program code that, when executed by the processor, causes the processor to filter the received movement data using a noise filter.

Another broad aspect is a non-transitory storage medium that, when executed by a processor, causes the processor to receive movement data of the subject comprising one or more of force magnitude information, force direction information and angular velocity information generated by the at least one of one or more accelerometers and one or more gyroscopes; categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement, associated with a level of attention of the subject.

Another broad aspect is a non-transitory storage medium that, when executed by a processor, causes the processor to receive movement data of the subject comprising one or more of force magnitude information, force direction information and angular velocity information generated by the at least one of one or more accelerometers and one or more gyroscopes; categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement; receive user input from the wearable device relating to attention of the subject; correlate time of the user input with time of the movement data that is categorized as fidgeting movement; and further categorize the fidgeting movement into unhelpful fidgeting or helpful fidgeting as a function of the user input that has the time of the user input correlated with the corresponding time of the movement data of the categorized fidgeting movement.

### BRIEF DESCRIPTION OF THE DRAWINGS

The invention will be better understood by way of the following detailed description of embodiments of the invention with reference to the appended drawings, in which:

FIG. 1 is a block diagram of an exemplary network for generating a drug prescription recommendation for improving attention of a subject;

FIG. 2A is a diagram of an exemplary artificial intelligence algorithm / artificial neural network for classifying movement data;

FIG. 2B is a first-half of a diagram illustrating exemplary steps performed for training an exemplary artificial intelligence model / artificial intelligence neural network for determining fidgeting from movement data;

FIG. 2C is the second-half of the diagram of FIG. 2B illustrating exemplary steps performed for training an exemplary artificial intelligence model / artificial intelligence neural network for determining fidgeting from movement data;

FIG. 2D is a diagram illustrating exemplary steps performed by an exemplary long short-term memory (LSTM) model;

FIG. 3 is a flowchart diagram of an exemplary method of generating a report of prescription recommendations as a function of fidget data and characteristic data of the subject;

FIG. 4 is a flowchart diagram of an exemplary method of training and validation of an artificial intelligence model for classifying movement data;

FIG. 5 is a flowchart diagram of an exemplary method of classifying movement data of the subject using a trained artificial intelligence model;

FIG. 6 is a flowchart diagram of an exemplary method for training an artificial intelligence model for retrieving subject profiles from a database based on data on a subject of interest;

FIG. 7 is a flowchart diagram of an exemplary method of retrieving profiles of users having received prescriptions for improving attention that match a query generated from data on a subject of interest;

FIG. 8 is a flowchart diagram of an exemplary method of refining the retrieval of profiles of users having received prescriptions for improving attention that match a query generated from data on a subject of interest;

FIG. 9 is a flowchart diagram of an exemplary method for training an artificial intelligence model for recommending a prescription to a subject; and

FIG. 10 is a flowchart diagram of an exemplary method of recommending a prescription to a subject for improving attention of the subject.

### DETAILED DESCRIPTION

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

### Definitions:

In the present disclosure, by “drumming”, it is meant a class of movement that involves repetitive hand motions alternating in a left-right, left-right fashion.

In the present disclosure, by “fidgeting movement”, refers to movement of an individual that is cyclical, repetitive that is not directed to a functional purpose (such as writing, typing, drawing, etc.). Fidgeting movement may include shuffling, wiggling or engaging in other ‘still or seated movements’, commonly seen in children and adults with Attention Deficit, Hyperactivity Disorder, also including but not limited to wrist and hand movements, and including hand flapping, hand-wringing and other stereotyped behaviors commonly associated with Autism stimming, that may affect the person's productive behavior and/or be related to their ability to quietly sit or stand and attend or focus on a given task.

In the present disclosure, by “jumping”, it is meant a class of movement that involves cyclical increases and decreases of vertical elevation of the torso, which may or may not involve an individual's feet leaving the ground.

In the present disclosure, by “rocking”, it is meant a class of fidgeting that involves cyclical gyrations of the torso, which could be in a fore and aft, or side-to-side motion, circular or swaying motion.

In the present disclosure, by “subject” or “patient”, it is meant a human. The term “subject” or “patient” should not bring on any limitations as to the sex or age.

In the present disclosure, by “tapping”, it is meant a class of fidgeting that involves repetitive, single-handed touching of a surface, typically led by a single digit or extremity (e.g. finger, foot/leg, etc.)

### Network for Providing Recommendations for Drug Prescription:

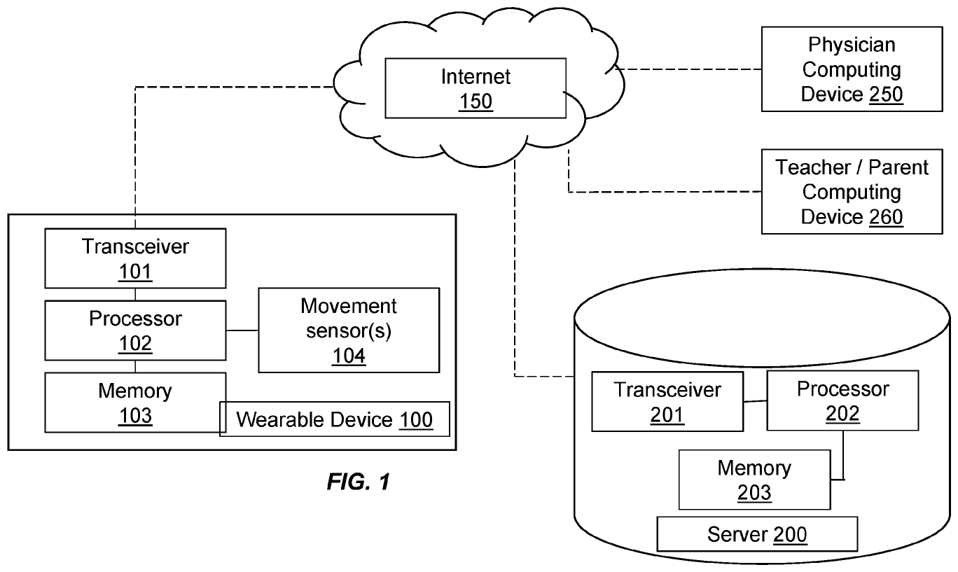

Reference is made to FIG. 1, illustrating an exemplary network for providing recommendations for drug prescription to improve attention of a subject, e.g. where the subject has ADHD, hyperactivity, etc., or is suspected to have ADHD, hyperactivity, etc.

The network includes a server 200 and one or more wearable devices 100 worn by one or more subjects whose attention is being monitored, the server 200 and the one or more wearable devices 100 in communication wirelessly, e.g., over the Internet 150.

The server 200, e.g. through transceiver 201, may be in communication with a physician computing device 250 and/or a teacher computing device 260 and/or a parent computing device 260.

The server 200 includes a processor 202, memory 203 and an input/output interface 201 (e.g. transceiver).

The processor 202 may be a programmable processor. In this example, the processor 202 is shown as being unitary, but the processor 202 may also be multicore, or distributed (e.g. a multi-processor).

The computer readable memory 203 stores program instructions and data used by the processor 202. The memory 203 may be non-transitory. The computer readable memory 203, though shown as unitary for simplicity in the present example, may comprise multiple memory modules and/or cashing. In particular, it may comprise several layers of memory such as a hard drive, external drive (e.g. SD card storage) or the like and a faster and smaller RAM module. The RAM module may store data and/or program code currently being, recently being or soon to be processed by the processor 202 as well as cache data and/or program code from a hard drive. A hard drive may store program code and be accessed to retrieve such code for execution by the processor 202 and may be accessed by the processor 202 to store fidget data on the subject, the trained artificial intelligence model for classifying movement data, characteristic data on the subject, queried profiles from the database, etc. The memory 203 may have a recycling architecture for storing, for instance, fidget data, prescription recommendations, etc., where older data files are deleted when the memory 203 is full or near being full, or after the older data files have been stored in memory 203 for a certain time.

The processor 202, the memory 203 and the I/O interface 201 may be linked via BUS connections.

The wearable device 100 collects movement data on the subject wearing the device. The wearable device transmits the movement data to the server 200 via an I/O interface 101 (e.g. transceiver). The wearable device 100 may be a wristband, an ankle bracelet, a headband, may be integrated into clothing such as the T-shirt of the subject, etc.

The wearable device 100 may also have a component to cause the wearable device 100 to vibrate, such as a direct current motor, a linear resonant actuator, etc.

The wearable device 100 includes a processor 102, memory 103, one or more motion sensors 104 (e.g. accelerometers, gyroscopes, etc.) and an I/O interface 101 (e.g. transceiver).

The processor 102 may be a programmable processor. The processor 102 may be part of a chip or microchip of the device 100.

The computer readable memory 103 stores program instructions and data used by the processor 102. The memory 103 may be non-transitory. The memory 103 may store program code for filtering out noise of the movement data generated by the one or more motion sensors 104.

The teacher computing device 260 and/or the parent computing device 260 may include a graphical user interface for enabling the teacher and/or the parent, respectively, to transmit feedback to the server 200 on the behavior of the subject wearing the wearable device 100 (i.e. the attention of the subject). This feedback report from either the parent or the teacher may be prompted by a questionnaire transmitted to same on a punctual basis.

The physician computing device 250 may receive the drug prescription recommendations generated by the server 200 for the subject wearing the device 100 as described herein. The physician may then take under advisement the drug prescription recommendations when prescribing a drug, or modifying a prescription of a drug, for a subject.

When the wearable device 100 transmits the movement data to the server 200 for analysis, the movement data may be sent along with metadata, wherein the metadata includes a code or identifier for the subject wearing the wearable device 100 in order for the generated drug recommendations to be associated with the proper subject, in embodiments where the server 200 is receiving movement data from a plurality of wearable devices 100, wherein a wearable device 100 is associated with a particular subject.

### Exemplary Method of Generating a Recommendation for Drug Prescription to Improve Attention:

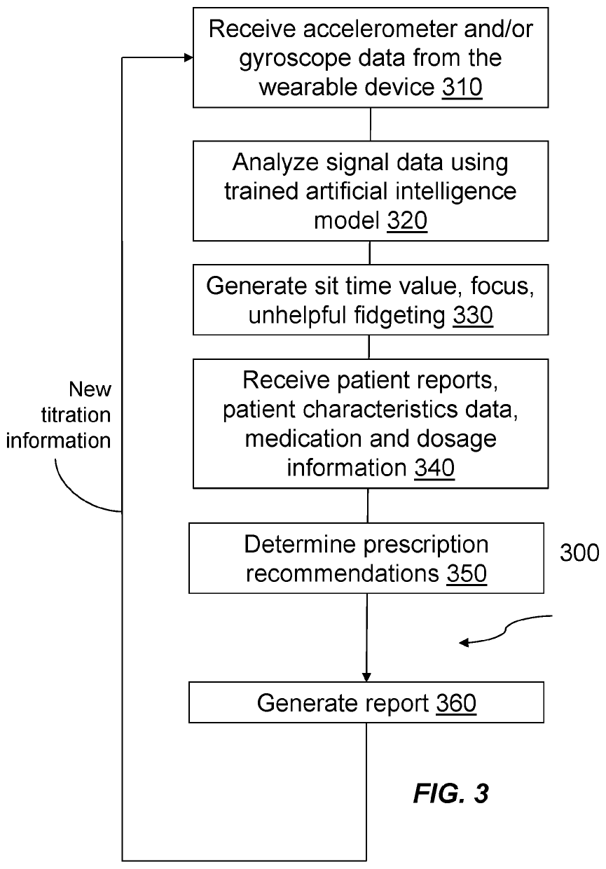

Reference is now made to FIG. 3, illustrating an exemplary method 300 for generating a recommendation for drug prescription to improve attention of a subject. For purposes of illustration, reference will be made to the elements of the network of FIG. 1. However, it will be understood that any other device 100, server 200 or other element illustrated in FIG. 1 may be used in accordance with the present teachings.

The method 300 includes receiving movement data (e.g. accelerometer and gyroscope data) from the wearable device 100 (e.g. from transceiver 101 to transceiver 201) at step 310 generated by the motion sensors (e.g. accelerometers) of the wearable device 100. The vibration data may be first filtered at the level of the wearable device 100 to filter out the noise (i.e. background noise of the collected data) prior to receipt by the server 200. In some examples, it will be understood that instead the server 200 may perform the filtering to remove the noise. The noise may be vibrations caused by, e.g., subtle trembling or shaking of the patient, the movement caused the heartbeat of the patient, etc.

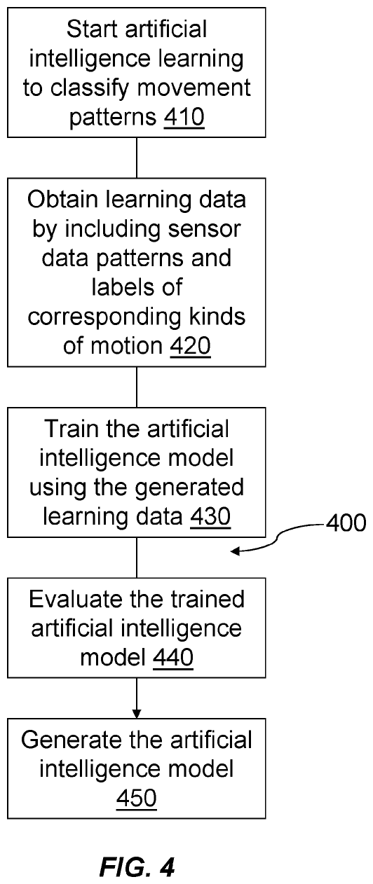

The received vibration data may then be analyzed using a trained artificial intelligence model at step 320. An exemplary method of training the artificial intelligence model is illustrated at FIG. 4 and explained herein.

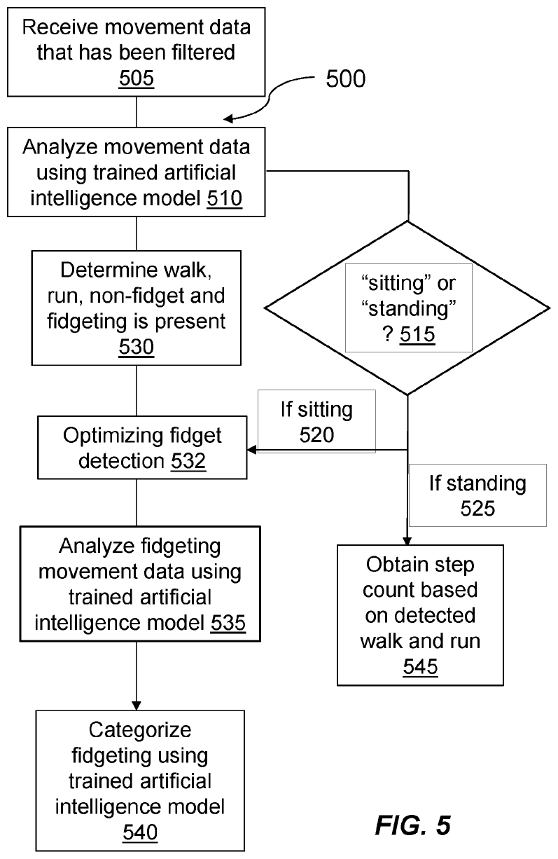

The analysis of the received vibration data is illustrated by the exemplary method 500 of FIG. 5.

The server 200 receives the movement data from the wearable device 100 that, in some examples, has been filtered to remove noise, at step 505.

The movement data is then analyzed using the trained artificial intelligence model, trained to classify the movement data at step 510. The classification is performed by analyzing the movement patterns over time. Changes in movement patterns signal a change in the type of movement pattern. The artificial intelligence model is trained to analyze the characteristics of the movement data. In the case of an accelerometer and/or gyroscope, the movement data includes information on the force and/or direction of the force and/or angular velocity over time. As such, changes in the direction of the force and/or angular velocity are also picked up by the sensors that are part of the wearable device 100. The force and direction of the force and/or angular velocity over time can be analyzed to provide information such as acceleration, amplitude of force, repetitive patterns, etc. The artificial intelligence model is trained to determine characteristics from the movement data, and compare same with studied samples of movement data to classify the movement data as a function of the attributes of the movement data. The pitch, force as a function of time, patterns of force over time can provide fingerprints for given kinds of movement that are recognized by the trained artificial intelligence model. Once recognized, the trained artificial intelligence model associates the movement data with a label (e.g. in metadata for the movement data, or by generating a value of time for a given classification of movement, e.g. associated with a given timestamp).

For instance, in some embodiments, the movement data is classified by the trained artificial intelligence model as being movement data generated when the subject was in the standing position or the sitting position. This additional classification step may improve the subsequent classification of movement data as “fidgeting” or “non-fidgeting”. The classification of movement data as “standing” or “sitting” may be determined through characteristics of the movement data, as stated above. The trained artificial intelligence model labels the movement data as being associated with the standing position or the sitting position at step 515, e.g. based on timestamps, where a portion of movement data is labeled as “standing”, another portion is labelled as “sitting”, etc.

As such, the movement data is then further labeled as fidgeting movement or non-fidgeting movement using the trained artificial intelligence model at step 530, by further studying the characteristics of the movement data.

For the movement data labeled as obtained in or associated with the sitting position at step 520, the sitting label can be used to optimize the fidget detection at step 532, e.g. by confirming that the movement data with the “fidget” label also includes the “stand” label.

Following a further analysis of the fidgeting movement data using an artificial intelligence model that is trained to classify the fidgeting movement data into classes of fidgeting at step 535, the fidgeting movement data may then be further classified by the trained artificial intelligence model under different forms of fidgeting at step 540 such as drumming, tapping, hopping, jumping, rocking, etc. Non-fidgeting movement data may be handwriting, hand raising, turning around in a seat to, e.g., speak to the teacher or to a classmate, etc.

In some embodiments, the categorizing of the data as “fidgeting” or “non-fidgeting” may be performed concurrently with the classification of data as “sitting” or “standing”. For instance, instead of the trained artificial intelligence model determining that the movement data is classified as “sitting” or “standing”, the movement data may be classified as “standing”, “non-fidgeting” or “fidgeting”, where labels for “fidgeting” and “non-fidgeting” are counted by the program code as “standing”, e.g. added to a value for time lapsed sitting by the patient.

In some embodiments, the trained artificial intelligence model may directly classify the movement data into the different categories of “fidgeting”, without going through the stratified process of first classifying as “standing” and “sitting”, and then “fidgeting” and “non-fidgeting”. The sub-categories of fidgeting are then counted by the program code as “fidgeting”, e.g. added to a value for time lapsed fidgeting for the patient.

In some examples, the fidgeting data may be further classified as “helpful fidgeting” or “unhelpful fidgeting” by the processor executing the program code stored in memory, such as the program code associated with the trained artificial intelligence module. Information is received on the subject regarding the attention of the subject (e.g. if the subject is on-task or off-task), this information including time data indicative of at what time the attention information is collected. The information received by the subject indicative of if the subject is on-task may be feedback information provided by the subject using, e.g., the wearable device. For instance, the wearable device may include one or more buttons, and/or a touchscreen display with one or more icons appearing on the display, that can be selected by the user indicative of if the user is on-task or off-task. The movement data that is classified as fidgeting is further classified as unhelpful fidgeting or helpful fidgeting by mapping the fidgeting movement data to the attention information as a function of time. If the subject is focused at the times where there is fidgeting data, then the fidgeting data is labelled as “helpful”. However, if the subject is unfocused at the times where the movement data is classified as “fidgeting”, then the fidgeting data is labelled as “unhelpful”. The distinction between “unhelpful fidgeting” and “helpful fidgeting” can provide further specificity as to the efficacy of a prescription, as fidgeting that helps the subject concentrate may not necessarily be a sign that the subject is suffering from a condition that affects the user's attention.

In some examples, the movement data may also be analyzed by the trained artificial intelligence model to generate an indicator value regarding hyperactivity (e.g. such as a hyperactivity score, or a value associated with presence of hyperactivity or a value associated with absence of hyperactivity). This indicator may be generated through an analysis of the movement data classified as “fidgeting” or “unhelpful fidgeting”, where the fidgeting or unhelpful fidgeting may be a sign of restlessness associated with hyperactivity.

The movement data may be associated with an identifier of the wearer of the wearable device (e.g. a name; a code identifying the individual, etc.) transmitting the movement data, and/or an identifier of the wearable device itself (e.g. serial number of the wearable device).

If the movement data was categorized as data obtained in the standing position by the trained artificial intelligence model at step 525, the movement data may be further categorized by the trained artificial intelligence model as “walking movement” and “running movement”.

Further analysis of the movement data using the trained artificial intelligence model or an algorithm searching for patterns in the movement data can determine the number of steps taken by the subject at step 545.

Returning to the method 300, the trained artificial intelligence model generates information and classification from the movement data (as described for method 500) that can be further used to determine the prescription for the subject as explained herein at step 330. This information may be a value for the time seated by the subject, the time standing by the subject, a time or frequency value for unhelpful fidgeting, the time spent carrying out one of the sub-classifications of fidgeting, etc.

Subject characteristic data is then received at step 340. The subject characteristic data may include the age of the subject, the weight, height, gender and/or ethnicity of the subject. It will be understood that other subject characteristic data may be received on the subject, such as a geographical location of the subject, underlying medical conditions, etc.

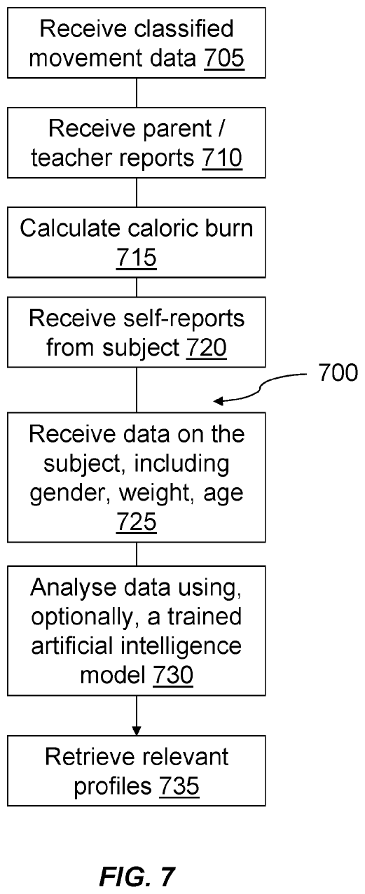

Reference is made to the exemplary method 700 of FIG. 7 to further illustrate exemplary steps leading to the generating of the recommended prescription.

The movement data classified by the trained artificial intelligence model is received at step 705 (e.g. fidgeting data; fidgeting time; kinds of fidgeting). Additional information associated with the classified movement data may be provided instead of, or in addition to, the classified movement data. Such additional information may be, for instance, the time spent by the patient carrying one or more of the type of fidgeting movement, the time spent standing, the time spent sitting, a ratio of fidgeting and non-fidgeting, etc.

The server 200 may also receive feedback provided by a parent and/or teacher using one or more computing devices 260 (e.g. observed attention of the subject) at step 710.

The caloric burn of the subject may also be calculated from the number of steps calculated during a given period (walking steps; running steps; as determined by the trained artificial intelligence model), as well as a value for the rate of calorie burn per step (or walking time, running time, respectively) associated with walking or running at step 715. A caloric burn may also be calculated when the patient is sitting (e.g. a product of time spent sitting and calories spent as a function of time seated).

The subject, e.g. (on their device 100) may also provide feedback as to their perceived level of attention during a time period or activity at step 720. The feedback may be provided through the wearable device on a punctual basis, where the wearable device solicits a response from the subject to, for instance, provide an indicator of the level of attention of the subject while performing a given task (e.g. in real time). The wearable device may provide a user input interface (e.g. a touchscreen) where the feedback request may be displayed as a question (e.g. “What is your level of focus now?”, or “Are you currently on-task or off-task?”). The user interface may also provide a selection of answer options, e.g., as icons (such as a “yes” icon and “no” icon). The user may be prompted to look at the wearable device by a signal, e.g., a vibration, a light, a sound, etc.

Characteristic data on the subject, as described, e.g., for step 340, is also obtained at step 725.

The caloric burn and/or classified movement data information and related movement information and/or received feedback (from the patient or an observer, such as a parent or a teacher) may be stored in memory, e.g., with a timestamp, in association with the patient, in order to gather information on the patient over a prolonged period. The stored information can be retrieved to compare data between different time periods, to provide an overall portrait of the patient, etc.

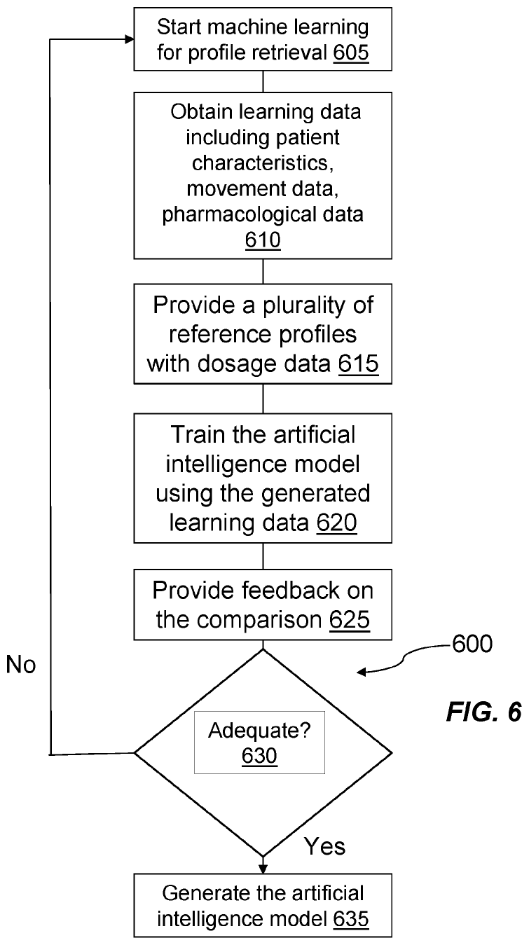

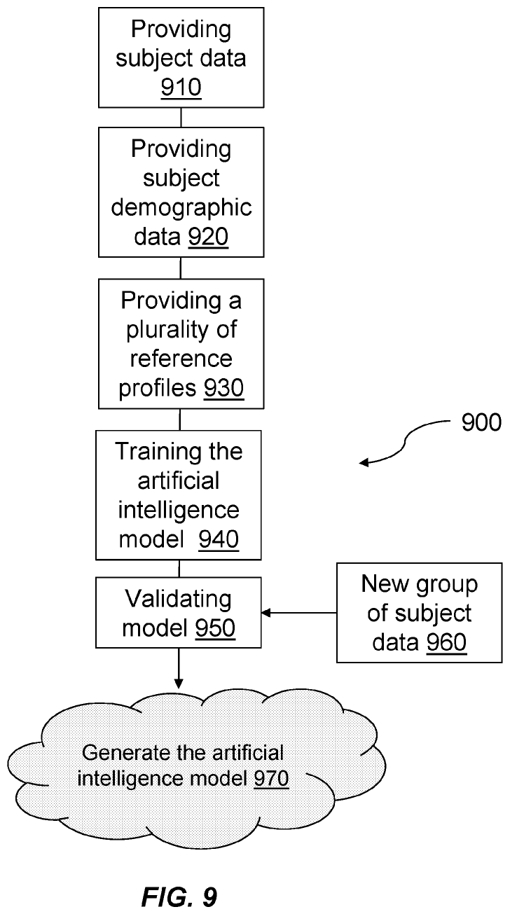

The received data can be analyzed and filtered to generate a query for retrieving profile data structures from a database at step 730. This analysis step may be performed by a trained artificial intelligence model, as illustrated by the exemplary method 600 of FIG. 6, or exemplary method 900 of FIG. 9.

A query is then generated for a database storing a plurality of user profile data structures, the query for retrieving one or more of the stored user profile data structures. Each user profile data structure is associated with a subject having been prescribed a drug for treating a condition that affects attention. The profile data structure may include information on the subject associated with the profile data structure, including, as entries, one or more of the following examples:
characteristic data: one or more of age, gender, height, weight, ethnicity, etc. of the subject;
the prescribed drug and the corresponding dosage, and any changes to the prescription over time;
relevant fidgeting data, movement data information (e.g. a ratio between fidgeting and non-fidgeting), caloric burn data, e.g., before and after prescription;
an indicator or value as to if the prescription improved the symptoms of the subject;
name or identifier of the subject;
identifier of the wearable device associated with the subject;
metadata acting as a legend of the types of data found in the profile data structure.

In some examples, the profile data structures that populate the database are of subjects that have shown a noticeable improvement in their symptoms between a time prior to and subsequent to taking the prescription. A value may be included in the profile data structure corresponding to the degree of improvement of the subject. In one example, this improvement value may be calculated by a processor executing program code to compare classified movement data from the time prior to and subsequent to taking the prescription. For instance, a value corresponding to a total amount of movement data time associated with unhelpful fidgeting taken from both a time prior to and subsequent to taking the prescription can be compared to one another to generate a ratio indicative of a chance in unhelpful fidgeting, and the value can be calculated from that ratio. In another example, this improvement value may be calculated by a processor executing program code to compare feedback information provided by the subject using the wearable device (e.g. by answering the punctual questions generated on a display of the wearable device prompting a response from the user, e.g. indicative that the user is or not on-task), between the time prior to and subsequent to taking the prescription.

The generated query contains one or more criteria for retrieving profile data structures that are relevant to the search. Using the metadata for each profile data structure, the profile data structures meeting the criteria of the query are selected (e.g. threshold value of fidget rate; gender; age; threshold value for average daily caloric burn; etc.) The selected profile data structures are retrieved by the server 200 at step 735.

The retrieved profile data structures are analyzed, the prescription information (selected drug, and, optionally, dosage) for generating the drug recommendations extracted from the profile data structures at step 350.

A report with the prescription recommendations is generated at step 360 (e.g. for transmission to a physician treating the patient). The report may include one or more drugs, and a dosage regimen for each drug.

The method 300 can be further repeated to verify if the prescribed drug at the set dosage improves the condition of the subject. The dosage information can be stored in memory (of server 200 or an external database) to be retrieved by the server 200 when assessing if the prescription improves the condition of the subject. The current prescription information on the subject can be added as a criterion when conducting the query to retrieve relevant profile data structures from the database for changing or refining the prescription regimen. If the drug at the current dosage is not as effective, another drug and/or dosage from the report with prescription recommendations may be selected.

Additionally, the wearable device may also collect data that can provide information on if the patient is experiencing one or more side effects from the medication.

Moreover, the movement data collected on the subject after prescription, when medicated, can be compared with movement data pre-prescription to determine if the condition of the subject is improving once the patient has received the medication. Feedback from the subject, and/or third parties such as the teacher and/or parent, can be further assessed to determine the efficacy of the prescription.

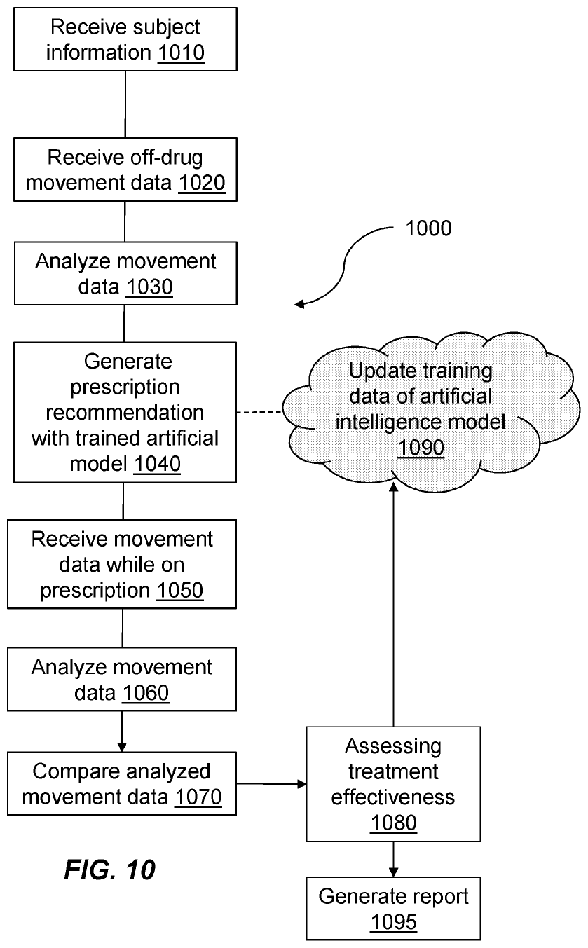

In another example, a prescription recommendation can be generated for a subject by performing method 1000, as illustrated in FIG. 10. Steps 1040-1090 may be performed by a trained artificial intelligence model, trained through exemplary method 600 of FIG. 6, or exemplary method 900 of FIG. 9.

Subject characteristic information on the subject may be received at step 1010. Such information may include, but is not limited to, the age of the subject, the gender of the subject, the weight of the subject, the height of the subject, the resting heart rate of the subject, the blood pressure of the subject, biomarker information on the subject, etc.

Movement data, generated by the wearable device worn by the subject, is received at step 1020. The movement data is collected while the subject is in a state where the subject is currently not on a prescription.

The movement data is analyzed at step 1030 to, e.g., generate fidgeting information, sitting and standing information, etc., using a trained artificial intelligence model as described herein (e.g. performing method 500).

The artificial intelligence model trained using method 600 or method 900 receives the analyzed movement data with the movement data classification, the subject characteristic data, and generates a prescription recommendation at step 1040. The prescription recommendation information includes a drug type, with, in some examples, a dosage regimen. In some examples, the trained artificial intelligence model may generate several prescription recommendations and/or several dosage regimens for each drug selected.

Further movement data, generated by the wearable device worn by the subject, is received at step 1050. The movement data is collected while the subject is following the prescription recommendation.

The further movement data, collected while the subject is following the prescription recommendation, is analyzed at step 1060 to, e.g., generate fidgeting information, sitting and standing information, etc., using a trained artificial intelligence model as described herein (e.g. performing method 500).

The analyzed movement data collected while the subject is following the prescription recommendation is compared to the analyzed movement data while the subject is not taking the prescription at step 1070 by the trained artificial model for generating a drug prescription.

Treatment effectiveness is assessed at step 1080. If the comparison yields a result that little to no attention improvement (or a reduction in hyperactivity, e.g., measured by a reduction in unhelpful fidgeting) is detected for the subject while on the prescription (e.g. a comparable or greater amount of fidgeting; a comparable or greater amount of unhelpful fidgeting; based on subject input indicating a lack of attention), then the result is processed by the trained artificial intelligence model, trained with method 600 or method 900, to update the trained artificial intelligence model 1090. A new prescription recommendation may be generated by the updated trained artificial model at step 1040, and steps 1040-1090 may be repeated.

A report on the attention level (e.g. improvement of the subject) may be generate by the system at step 1095.

### Exemplary Method of Training an Artificial Intelligence Model for Classifying Movement Data:

Reference is now made to FIG. 4, illustrating an exemplary method for training an artificial intelligence model for classifying movement data into movement associated with a seated position or a standing position. In the case of movement data associated with a seated position, the artificial intelligence model may be further trained to distinguish between fidgeting movement and non-fidgeting movement, as well as classify the fidgeting movement into subtypes.

Artificial intelligence learning starts at step 410 to classify movement patterns.

Learning data for feeding to the model is generated at step 420. The learning data includes samples of movement data (e.g. the movement data generated by one or more accelerometers and/or gyroscopes corresponding to the ones of the wearable device 100). The samples of movement data include directional force data as a function of time (e.g. along each of three axes, x, y and z, for each of the accelerometers and/or gyroscopes), where the changes in directional force and/or angular velocity over time generate patterns for the kind of movement. The movement data samples are associated with labels of movement. For instance, for training the artificial intelligence model to recognize different levels of movement data:
a first set of samples may be associated with a standing position or a sitting position of a user, where the appropriate labels are provided and fed to the model. The samples may originate from a plurality of individuals in order to sensitive the model to differences in movement of different individuals for a “standing” position and a “sitting” position;
a second set of samples may be associated with movement data when the user is in a sitting position, namely to determine if the user is fidgeting or not-fidgeting while seated. The labels may be “fidgeting” or “not-fidgeting”. The samples may originate from a plurality of individuals in order to sensitive the model to differences in movement of different individuals;
a third set of samples may be associated with different forms of fidgeting. Labels and associated samples may be provided for, e.g., “drumming”, “tapping”, “jumping” and “rocking”, etc. The samples associated with each label may originate from a plurality of individuals in order to sensitive the model to differences in movement of different individuals;
a fourth set of samples may be associated with standing positions, where the samples and the labels may be for “running”, “walking” or “standing”. The samples may originate from a plurality of individuals in order to sensitive the model to differences in movement of different individuals.

The pairs of the samples and the labels are then fed to the artificial intelligence model to generate learning data at step 430, where the artificial intelligence model identifies common patterns in the changes of force over time between the different samples of movement data for a same classification (as determined by their labels), and distinguishes patterns or characteristics between samples of different classifications (as determined by their labels).

At step 430, artificial intelligence (AI) algorithms may include, but are not limited to CNN, and Long short-term memory (LSTM). The AI algorithm selection may depend on which model can give the desired results.

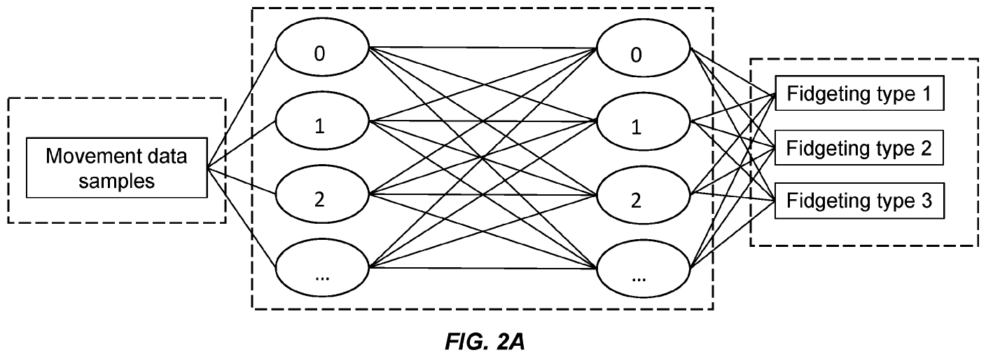

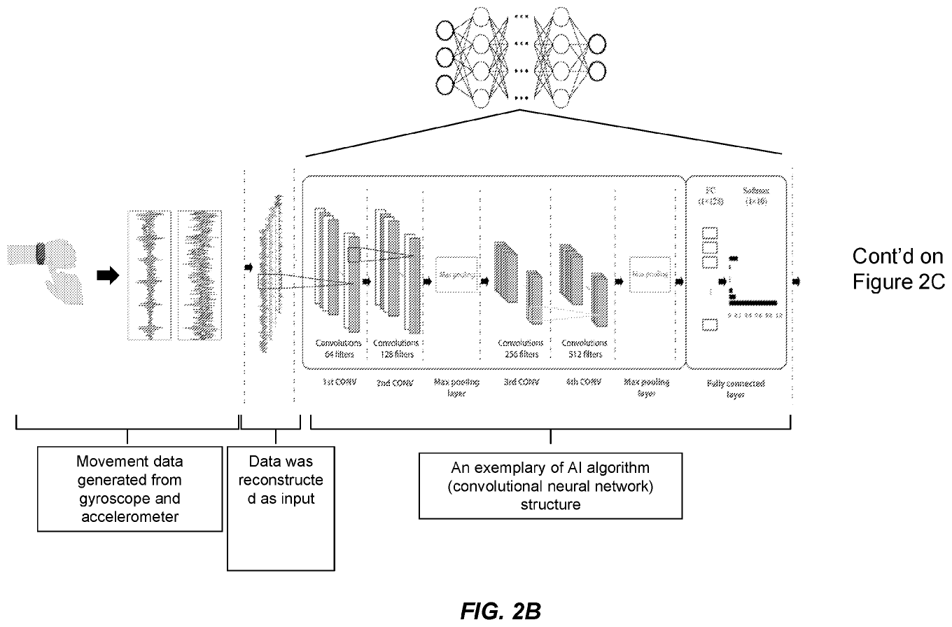

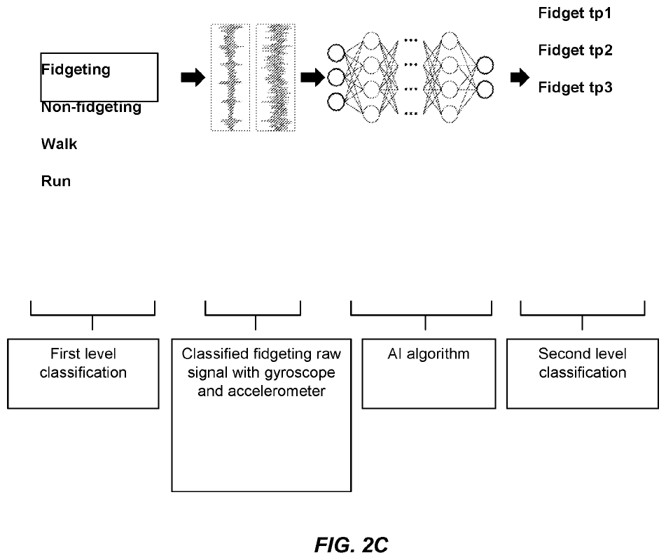

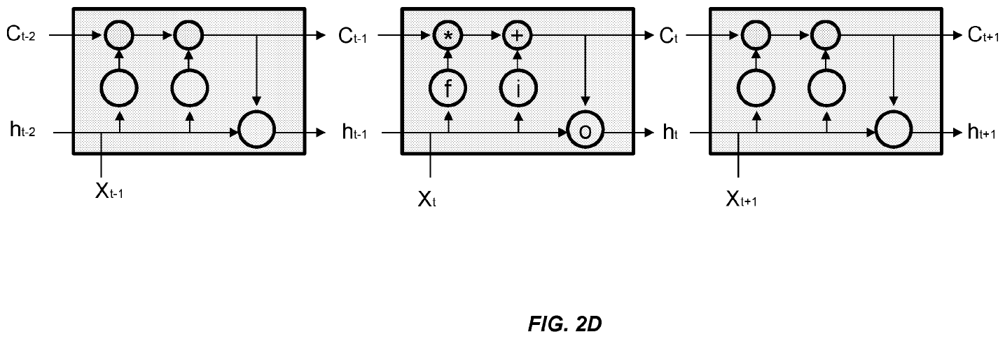

At step 430, 1D convolutional neural networks (CNNs) may be used for recognizing fidgeting and other motions based on 6-axis accelerometer and gyroscope data. CNNs may be trained via back-propagation through a sequence of layers; each layer will transform one volume of activation to another through a differentiable function. The example of CNNs architecture contained Input layer (INPUT), convolutional layer (CONV), Pooling Layer (POOL) and Fully-Connected Layer (FC). An example of architecture of CNNs is shown in FIG. 2B. For the INPUT, a series of fixed time-length data explained above may be used as input (e.g. each containing 6-axes gyroscope and accelerometer data in timestamps of a certain length). The signal may be further processed by calculating Frobenius and using a Fast Fourier transform (FFT) as the proper format of training dataset. In the CONV layers, transformed data may be piled as a sequence of rows as input. A certain number of filters with a certain kernel size and stride may be set in each CONV layer. The activation function used may be ReLU. The dropout rate may be 50% after each CONV and max-pooled features may be used to prevent overfitting. The multi-layer CNNs may then be followed with a flatten layer and fully connected layers. Softmax layer may be placed as an output layer of fully connected layers to predict the final classification of the input data. The optimizer may be Adam. CNNs maybe used but not limited to detect fidgeting, walk, run, steps, sitting/standing, sit/stand.

At step 430, Long short-term memory (LSTM), dealing with sequential data, may be further used to support detecting sitting and standing to optimize the fidgeting result.

At step 430, a Long short-term memory (LSTM) maybe be used. An example of the

architecture of LSTM is shown in FIG. 2D. In some examples, the output (sitting/standing) for a specific time step may be predicted from the input from the previous model that consists of the last n consecutive time steps processed sequentially. In the LSTM model, it has a cell state or cell memory cₜ where information can be stored, and gates that control the information flow within the LSTM cell (shown as 3 encircled letters in FIG. 2d). The first gate, f, is the forget gate, controlling which elements of the cell state vector cₜ₋₁ will be forgotten to a certain degree. An updated vector for the cell state, c~t, is computed from the current input (xₜ) and the last hidden state (hₜ₋₁). Information it is used to update the cell state. The third and the last gate is the output gate, controlling the information of the cell state cₜ which flows into the new hidden state hₜ. In the LSTM model, the LSTM layer may include a certain number of learnable input-hidden weights, the learnable hidden-hidden weights, the learnable input-hidden bias. The dense layer may include the Weight matrix and the bias layer. It is in particular the cell state (cₜ) that allows for an effective learning of long-term dependencies. cₜ interacts with the remaining LSTM model, storing information unchanged over a long period of time steps. The length of the cell and hidden state vectors in the LSTM can be chosen depending on the best result. A LSTM model may be used with a certain layer, associated with a certain input size multiply a certain hidden size. The Weight matrix may be the learnable input-hidden weights of a layer, with a certain shape and input size. The learnable hidden-hidden weights may correspond to the same layer. There may be learnable input-hidden bias with a certain shape multiply a certain hidden layer. A weight matrix and bias for dense layer may also be applied.

The trained artificial intelligence model may then be validated with one or more of the following four common metrics: accuracy, recall, precision and F-measure for model performance evaluation, by feeding the model with another series of unlabeled movement data that is separate from the training dataset, where their corresponding labels are known by the tester, and will be verified if the trained artificial intelligence model can accurately classify each of the samples at step 440 in accordance with the expected results.

Once the trained artificial intelligence model has been evaluated to be adequately trained, the trained artificial intelligence model is generated at step 450.

Exemplary Method of Training an Artificial Intelligence Model for Retrieving Profile Data Structures from a Database for Prescription Recommendation:

Reference is now made to FIG. 6, illustrating an exemplary method 600 of training an artificial intelligence model for retrieving profile data structures from a database for prescription recommendation.

Learning of the artificial intelligence model is started at step 605.

Datasets corresponding to one or more sample subjects are fed to the model at step 610. A dataset may include one or more of classified movement data, data associated thereto or derived therefrom (e.g. ratio between fidgeting/non-fidgeting; time spent carrying out different sub-categories of fidgeting), caloric burn, subject characteristics (e.g. age, gender, weight), as well as if the subject is currently prescribed a drug.

The model may then be provided with a plurality of profile data structures from which to query at step 615.

The artificial intelligence model is then trained at step 620 to generate learning data by correctly associating a dataset of the sample subjects with one or more profile data structures of the plurality of profile data structures found in the database. Exemplary queries may be generated by the model based on the association, where the queries include relevant criteria determined from the data categories found in the profile data structures and the data categories available for a given sample dataset.

The artificial intelligence model is then provided with a second set of sample datasets (where expected target profile data structures from the plurality of profile data structures are known to the tester but not provided to the model). The queries generated by the artificial intelligence model and the retrieved profile data structures are monitored by the tester. Feedback is provided to the artificial intelligence model as a function of the expected results (e.g. the retrieved profile data structures) at step 625. The artificial intelligence model adapts, e.g. through a dynamic model or data optimization, its queries as a function of the feedback received.

If the artificial intelligence model is adequate at step 630 (e.g. as determined by the test sets of sample datasets), then the artificial intelligence model may be generated at step 635. If not, any one of steps 605 and following may be repeated to further train the model.

Moreover, the profile data structures may include data (e.g. a value) indicative of if the treatment using a specific drug (with, optionally, a defined dosage) was effective at providing relief for a particular patient. In some examples, the database may include only profile data structures for patients for which the prescription showed a level of improvement. The profile data structure may include an indication as to the level of improvement acquired as a result of the prescription.

When populating the database with profile data structures including data indicative of a level of relief of the condition provided by the treatment, this data may be determined from feedback given by the patient (e.g. using the wearable device 100) that the treatment is, or is not, resulting in symptom improvement (e.g. improvement to concentrate on certain tasks) using, e.g., a user input interface of the wearable device 100. The data indicative of a level of relief provided by the treatment can also be determined from the movement data, classified using the trained artificial intelligence model. The classified movement data can separate helpful fidgeting from unhelpful fidgeting, where a decrease in the ratio of unhelpful fidgeting to helpful fidgeting before and after treatment is indicative of effectiveness of the treatment. Helpful fidgeting is fidgeting that does not distract a user from completing a task on-hand (where the user is on-task). On the other hand, unhelpful fidgeting is fidgeting that distracts the user from completing a task, or that is an indicator that the user has lost their attention in completing a specific task (where the user is off-task).

Based on a query is instead performed by providing the untrained artificial model with a large plurality of profile data structures, each including data on the effectiveness of the treatment (in some examples, the profile data structures provided to the artificial intelligence model only correspond to patients with whom a treatment plan effectively relieved symptoms). In some examples, the profile data structures may include for the given patient data entries for a plurality of treatments provided to the patient, each associated with data associated with effectiveness of that treatment for that patient, measured from input as described herein (e.g. from the movement data; feedback received from the patient using the wearable device, from a parent using a computing device, a teacher using a computing device, etc.) The profile data structures also include a list of data entries providing information on the patient's traits, such as, but not limited to: height, gender, weight, comorbidities, genetic information such as recessive mutations and expression of certain genes, body temperature, heart rate, blood sugar level, Galvanic skin response, hair colour, eye colour, body mass index, fidgeting information, physiological anomalies such as length ratio of fingers, etc. By providing the artificial intelligence model with sufficient data on a sufficiently large number of characteristics of a particular patient, and then by providing the artificial intelligence model with a sufficiently large quantity of profile data structures, the artificial intelligence model identifies the traits (as defined by the data for those traits) associated with a patient that are common among patients that have experienced a positive response to a specific form of treatment (e.g. particular drug, at a particular dosage). These traits are indicative that a specific form of treatment would be effective for a given patient. The larger the number of trait data entries, the greater the odds that the artificial intelligence model is sufficiently trained to locate relevant trait values to determine which trait values correspond to effectiveness of treatment.

Once the artificial intelligence model is trained, the trained artificial intelligence model receives a query including a list of trait values, corresponding to traits, for the given patient that is to receive a treatment recommendation. The trained artificial intelligence model identifies amongst the trait values those that are relevant for the purpose of determining a specific form of treatment, analyzes those trait values, and retrieves profile data structures of patients that match those trait values, the profile data structures associated with an effective treatment when given to the patient corresponding to the profile data structure.

Steps 350 and 360 may then be performed, as in exemplary method 300.

Reference is made to FIG. 9, illustrating another exemplary method 900 of training an artificial intelligence model for generating a prescription recommendation.

A set of subject data for one or more subjects is provided at step 910. For each subject, the subject data may include one or more of the following, each provided pre-drug prescription and post-drug prescription:
patient characteristic information: information on the characteristics of the subject, such as the age of the subject, the gender of the subject, the weight of the subject, the height of the subject, the resting heart rate of the subject, the blood pressure of the subject, biomarker information on the subject, etc.
movement data: movement data on the subject collected by the wearable device as described herein, wherein fidgeting information, and/or sitting and standing information, etc., may have been derived from the movement data, as explained herein;
pharmacological data: the type of medication prescribed to the patient, including dosage information (e.g. quantity, frequency of intake, etc.)

Demographic information on the subject may be provided at step 920. The demographic information may include information on where the subject falls within a class of individuals with similar traits (e.g. the medication prescribed to the subject is also prescribed to 30% of individuals with the age of the subject).1

The model may then be provided with a plurality of profile data structures with dosage information at step 930.

The artificial intelligence model is then trained at step 940 to generate learning data by correctly associating one or more of the sample subjects with one or more of the reference profile data structures.

The artificial intelligence model is then provided with a second set of subject data without prescription information at step 960. The artificial intelligence model is then prompted to generate a prescription recommendation for the sample subject based on the subject data.

The artificial intelligence model is then validated at step 950. The artificial intelligence model may be validated by verifying if the generated prescription recommendation is appropriate based on the subject information. The verification may be performed by a user, reviewing the generated prescription information and the subject data.

If the artificial intelligence model is adequate at step 950 (e.g. as determined by the test sets of sample datasets), then the artificial intelligence model may be generated at step 970. If not, any one of steps 910 and following may be repeated to further train the model.

### Query for Retrieving Profile Data Structures:

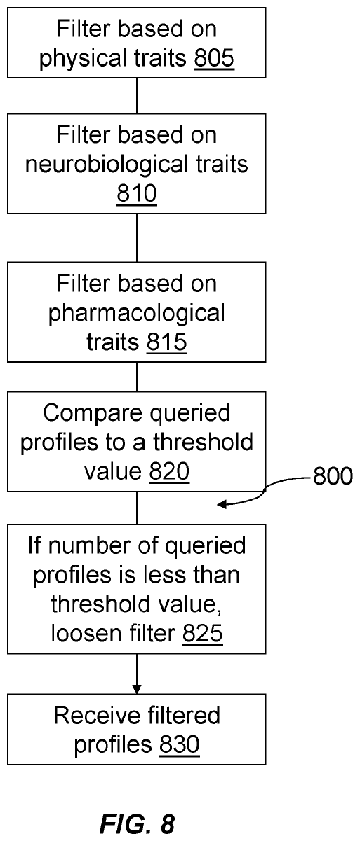

The query for retrieving one or more profile data structures may be tailored as described in exemplary method 800, illustrated at FIG. 8.

The query may provide criteria that filter based on physical traits (e.g. gender; age; height; weight; ethnicity; etc.) at step 805.

The query may provide criteria that filter based on neurobiological traits (e.g. attention span; fidget rate; average of time seated vs. standing; diagnosis; etc.) at step 810. Some of these criteria may be a threshold value for the purpose of the query, such as the attention span.

The query may provide criteria that filter based on pharmacological traits (e.g. type of drug; dosage; outcome/result; etc.) at step 815.

Once the query has retrieved a number of profile data structures, the number of retrieved queried data structures may be compared to a threshold value for a minimal number of profile data structures to retrieve.

If the number of retrieved profile data structure is less than the threshold value, the filtering of profile data structure when querying may be loosened by removing one or more of the criteria. The removal of the criteria may be performed based on an order of their corresponding type (e.g. starting first with the pharmacological traits; followed by the neurobiological traits; then the physical traits).

A new set of profile data structures may then be received at step 830 based on the new query. If the number of received profile data structure is still lower than the threshold value, further removal of one or more criteria of the query may be performed, followed by a transmission of the new query.

FIG. 2 is an exemplary diagram of an artificial intelligence model (using artificial neural network as an example) according to an embodiment of the present disclosure for classifying movement data.

Many artificial intelligence models with a variety of Machine Learning algorithms may be used to classify data. Representative examples of such machine learning algorithms for data classification include a decision tree, a Bayesian network, a support vector machine (SVM), an artificial neural network (ANN), etc.

ANNs may refer generally to models that have artificial neurons (nodes) forming a network through synaptic interconnections, and acquire problem-solving capability as the strengths of synaptic interconnections are adjusted throughout training.

An ANN may include a number of layers, each including a number of neurons. In addition, the ANN can include the synapse for connecting between neuron and neuron.

ANNs include, but are not limited to, network models such as a deep neural network (DNN), a recurrent neural network (RNN), a bidirectional recurrent deep neural network (BRDNN), a multilayer perceptron (MLP), and a convolutional neural network (CNN).

An ANN may be classified as a single-layer neural network or a multi-layer neural network.

A single-layer neural network may include an input layer and an output layer.

In some examples, a multi-layer neural network may include an input layer, one or more hidden layers, and an output layer.

An input layer is a layer that accepts external data. The number of neurons in the input layer is equal to the number of input variables. The hidden layer is disposed between the input layer and the output layer and receives a signal from the input layer to extract the characteristics to transfer it to the output layer. The output layer receives a signal from the hidden layer and outputs an output value based on the received signal. Input signals between the neurons are summed together after being multiplied by corresponding connection strengths (synaptic weights). If this sum exceeds a threshold value of a corresponding neuron, the neuron can be activated and output an output value obtained through an activation function.

In the meantime, a deep neural network with a plurality of hidden layers between the input layer and the output layer may be the most representative type of ANN which enables deep learning, which is one machine learning technique.

A machine learning model (e.g., ANN) can be trained by using training dataset. Herein, the training can mean a process of determining a parameter of the ANN in order to achieve the objects such as classification, regression, clustering, etc. of input data. As a representative example of the parameter of the ANN, there can be a weight given to a synapse or a bias applied to a neuron.

Examples of unsupervised learning (a subcategory of machine learning models) include but are not limited to clustering and independent component analysis.

Examples of artificial neural networks using unsupervised learning include, but are not limited to, a generative adversarial network (GAN) and an autoencoder (AE).

Examples of supervised learning (a subcategory of machine learning models) include Supervised Vector Machine and Regression Tree.

Examples of artificial neural networks using supervised learning include convolutional neural networks (CNNs), recurrent neural networks (RNNs) and Long short-term memory (LSTM).

An exemplary deep neural network is a convolution neural network.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described

above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is:

1. A method for identifying fidgeting of a subject from movement data gathered by a wearable device while worn by a subject, the wearable device including at least one of one or more accelerometers and one or more gyroscopes, the fidgeting related to a level of attention of the subject, comprising:
receiving movement data of the subject comprising one or more of force magnitude information, force direction information and angular velocity information generated by the at least one of one or more accelerometers and one or more gyroscopes;
categorizing the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement, associated with a level of attention of the subject.

2. The method as defined in claim 1, further comprising:
filtering the movement data as being associated with a sitting state of the subject, wherein the movement data associated that is categorized as associated with fidgeting movement or non-fidgeting movement is the movement data associated with the sitting state.

3. The method as defined in claim 2, wherein the filtering is performed using a trained long short-term memory artificial intelligence model.

4. The method as defined in any one of claims 1 to 3, further comprising:
further categorizing the movement data with a label of fidgeting movement as a sub-type of fidgeting using a previously trained artificial intelligence model to generate fidgeting data that is indicative as to a lack of attention of the subject, wherein the previously trained artificial intelligence model is a movement classifying engine that is trained with learning data including samples of fidgeting-labeled movement data that is matched with labels for sub-categories of fidgeting movement.

5. The method as defined in claim 4, wherein the labels for sub-categories of fidgeting movement include drumming and tapping.

6. The method as defined in any one of claims 1 to 5, further comprising:
receiving feedback from the subject on a level of attention of a subject regarding a task;
correlating the received feedback from the subject with the movement data that is labelled as fidgeting movement; and
categorizing movement data that is labelled as fidgeting movement, following the correlation, as unhelpful or helpful fidgeting movement based on an indication that the subject is on-task or off-task from the received feedback.

7. The method as defined in any one of claims 1 to 6, wherein the received movement data was filtered using a noise filter prior to receipt.

8. A method for identifying fidgeting of a subject from movement data gathered by a wearable device while worn by a subject, the wearable device including at least one of one or more accelerometers and one or more gyroscopes, the fidgeting related to a level of attention of the subject, comprising:
receiving movement data of the subject comprising one or more of force magnitude information, force direction information and angular velocity information generated by the at least one of one or more accelerometers and one or more gyroscopes;
categorizing the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement;
receiving user input from the wearable device relating to attention of the subject;
correlating time of the user input with time of the movement data that is categorized as fidgeting movement; and
further categorizing the fidgeting movement into unhelpful fidgeting or helpful fidgeting as a function of the user input that has the time of the user input correlated with the corresponding time of the movement data of the categorized fidgeting movement.

9. The method as defined in claim 8, further comprising:
filtering the movement data as being associated with a sitting state of the subject, wherein the movement data associated that is categorized as associated with fidgeting movement or non-fidgeting movement is the movement data associated with the sitting state.

10. The method as defined in claim 8 or claim 9, wherein the user input is indicative of the subject being on-task or off-task.

11. The method as defined in any one of claims 8 to 10, wherein the received movement data was filtered using a noise filter prior to receipt.

12. The method as defined in any one of claims 8 to 11, further comprising filtering the received movement data using a noise filter.

13. A system for identifying fidgeting of a subject from movement data gathered by a wearable device while worn by a subject, the wearable device including at least one of one or more accelerometers and one or more gyroscopes, the fidgeting related to a level of attention of the subject, comprising:
a processor;
memory comprising program code that, when executed by the processor, cause the processor to:
receive movement data of the subject comprising one or more of force magnitude information, force direction information and angular velocity information generated by the at least one of one or more accelerometers and one or more gyroscopes;
categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement, associated with a level of attention of the subject.

14. The system as defined in claim 13, wherein the memory further comprises program code that, when executed by the processor, causes the processor to filter the movement data as being associated with a sitting state of the subject, wherein the movement data associated that is categorized as associated with fidgeting movement or non-fidgeting movement is the movement data associated with the sitting state.

15. The system as defined in claim 14, wherein the filtering is performed using a trained long short-term memory artificial intelligence model.

16. The system as defined in any one of claims 13 to 15, wherein the memory further comprises program code that, when executed by the processor, causes the processor to further categorize the movement data with a label of fidgeting movement as a sub-type of fidgeting using a previously trained artificial intelligence model to generate fidgeting data that is indicative as to a lack of attention of the subject, wherein the previously trained artificial intelligence model is a movement classifying engine that is trained with learning data including samples of fidgeting-labeled movement data that is matched with labels for sub-categories of fidgeting movement.

17. The system as defined in any one of claims 13 to 16, wherein the memory further comprises program code that, when executed by the processor, causes the processor to:
receive feedback from the subject on a level of attention of a subject regarding a task;
correlate the received feedback from the subject with the movement data that is labelled as fidgeting movement; and
categorize movement data that is labelled as fidgeting movement, following the correlation, as unhelpful or helpful fidgeting movement based on an indication that the subject is on-task or off-task from the received feedback.

18. A system for identifying fidgeting of a subject from movement data gathered by a wearable device while worn by a subject, the wearable device including at least one of one or more accelerometers and one or more gyroscopes, the fidgeting related to a level of attention of the subject, comprising:
a processor;
memory comprising program code that, when executed by the processor, cause the processor to:
receive movement data of the subject comprising one or more of force magnitude information, force direction information and angular velocity information generated by the at least one of one or more accelerometers and one or more gyroscopes;
categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement;
receive user input from the wearable device relating to attention of the subject;
correlate time of the user input with time of the movement data that is categorized as fidgeting movement; and
further categorize the fidgeting movement into unhelpful fidgeting or helpful fidgeting as a function of the user input that has the time of the user input correlated with the corresponding time of the movement data of the categorized fidgeting movement.

19. A non-transitory storage medium that, when executed by a processor, causes the processor to:
receive movement data of the subject comprising one or more of force magnitude information, force direction information and angular velocity information generated by the at least one of one or more accelerometers and one or more gyroscopes;
categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement, associated with a level of attention of the subject.

20. A non-transitory storage medium that, when executed by a processor, causes the processor to:
receive movement data of the subject comprising one or more of force magnitude information, force direction information and angular velocity information generated by the at least one of one or more accelerometers and one or more gyroscopes;
categorize the movement data as associated with fidgeting movement or non-fidgeting movement using a previously trained artificial intelligence model to generate fidgeting data that is indicative as to a lack of attention of the subject, wherein the previously trained artificial intelligence model is a movement classifying engine that is trained with learning data including samples of movement data that is matched with labels for fidgeting movement or non-fidgeting movement;
receive user input from the wearable device relating to attention of the subject;
correlate time of the user input with time of the movement data that is categorized as fidgeting movement; and
further categorize the fidgeting movement into unhelpful fidgeting or helpful fidgeting as a function of the user input that has the time of the user input correlated with the corresponding time of the movement data of the categorized fidgeting movement.
